(19) United States
(12) Patent Application Publication
(10) Pub. No.: US 2025/0331826 A1
(43) Pub. Date: Oct. 30, 2025

(54) ULTRASOUND DIAGNOSTIC EQUIPMENT WITH IMAGING PARAMETERS ADJUSTMENT USING COMMENTS ENTERED BY OPERATOR

(71) Applicant: GE Precision Healthcare LLC, Waukesha, WI (US)

(72) Inventor: Shunichiro Tanigawa, Hino (JP)

(21) Appl. No.: 19/190,454

(22) Filed: Apr. 25, 2025

(30) Foreign Application Priority Data
Apr. 25, 2024 (JP) 2024-071812

(51) Int. Cl. A61B 8/00; A61B 8/06; A61B 8/08; G06T 7/62; G16H 30/40

(52) U.S. Cl. CPC A61B 8/5223; A61B 8/06; A61B 8/0891; A61B 8/468; A61B 8/488; A61B 8/54; G06T 7/62; G16H 30/40; G06T 2207/10132

(57) ABSTRACT

An ultrasonic diagnostic device including one or more processors for controlling operation of the ultrasonic diagnostic device for acquiring a second ultrasonic image of the same examination site as a first ultrasonic image based on an annotation added to the first ultrasonic image.

### CROSS-REFERENCE TO RELATED APPLICATION

This application claim priority to Japanese Patent Application No. 2024-071812, which was file on Apr. 25, 2024 at the Japanese Patent Office. The entire contents of the above-listed application are incorporated by reference herein in their entirety.

### TECHNICAL FIELD

The present disclosure relates to an ultrasonic diagnostic device capable of adding annotations to ultrasonic images, and a storage medium containing commands to be executed by the ultrasonic diagnostic device.

### BACKGROUND

In an ultrasonic examination, after an ultrasonic image is generated by scanning an examination site of a subject, another type of ultrasonic image may be generated by scanning the same examination site again. For example, an examination site of a subject may be scanned to generate a B-mode image, and then the same examination site may be scanned again to generate a color Doppler image. In this case, the user may set a condition for generating the color Doppler image (for example, a value of a parameter for adjusting the image quality of the color Doppler image) with reference to an organ or the like depicted in the B-mode image.

### SUMMARY

According to a first aspect of the disclosure, an ultrasonic diagnostic device includes one or more processors for controlling operation of the ultrasonic diagnostic device for acquiring a second ultrasonic image of the same examination site as a first ultrasonic image based on an annotation added to the first ultrasonic image.

According to a second aspect of the disclosure, a non-transitory computer readable storage medium in which a command is stored, wherein the command, when executed by one or more processors, causes the one or more processors to execute controlling an operation of the ultrasonic diagnostic device for acquiring a second ultrasonic image of the same examination site as that of the first ultrasonic image based on an annotation added to the first ultrasonic image.

### BRIEF DESCRIPTION OF THE DRAWINGS

FIG. 1 A block diagram of an ultrasonic diagnostic device according to an embodiment.

FIG. 2 A diagram illustrating one example of a user interface 10.

FIG. 3 A diagram illustrating the flow in a first embodiment.

FIG. 4 An explanatory diagram of an annotation 31 added to an ultrasonic image 21.

FIG. 5 A diagram illustrating one example of the flow of step ST20.

FIG. 6 A schematic diagram of an ultrasonic image 22 displayed on a display unit 8.

FIG. 7 An explanatory diagram of a method of specifying the meaning of an annotation.

FIG. 8 An explanatory diagram of step ST23.

FIG. 9 An explanatory diagram of step ST24.

FIG. 10 A diagram schematically illustrating a color Doppler image 71 displayed on the display unit 8.

FIG. 11 A diagram illustrating one example of the flow of step ST30.

FIG. 12 A diagram illustrating one example of a Doppler window 81.

FIG. 13 An explanatory diagram of step ST32.

FIG. 14 A diagram illustrating the flow in a second embodiment.

FIG. 15 A diagram schematically illustrating an ultrasonic image 23.

FIG. 16 A diagram illustrating one example of the flow of step ST70.

FIG. 17 An explanatory diagram of step ST73.

FIG. 18 A diagram illustrating a position where the diameter of the aorta is measured.

FIG. 19 A schematic diagram of one example of measurement results.

FIG. 20 A diagram illustrating one example of the flow of step ST80.

FIG. 21 A schematic diagram of an ultrasonic image 25 displayed on the display unit 8.

FIG. 22 An explanatory diagram of step ST87.

FIG. 23 An explanatory diagram of step ST88.

FIG. 24 A diagram schematically illustrating a color Doppler image 72 displayed on the display unit 8.

FIG. 25 is an explanatory diagram of step ST90.

FIG. 26 A schematic diagram of an operation console of the ultrasonic diagnostic device 1 according to a third embodiment.

FIG. 27 One example of a flowchart of measurement steps in the third embodiment.

### DETAILED DESCRIPTION

In some cases, it is necessary for a user to perform an operation such as checking a site depicted in a B-mode image acquired in advance. The user may also need to manually input conditions for generating the color Doppler image. Therefore, when a condition for generating a color Doppler image is set, there is a problem in that work burden on the user increases.

Therefore, there is demand for art capable of reducing work burden on the user in a case where an ultrasonic image of an examination site of a subject is generated and then another ultrasonic image of the same examination site is generated.

According to the present disclosure, the operation of the ultrasonic diagnostic device for acquiring the second ultrasonic image of the same examination site as the first ultrasonic image is controlled based on the annotation added to the first ultrasonic image. The annotation is added by a user of the ultrasonic diagnostic device. Therefore, even in a case where it is difficult for the user to visually recognize the examination site of the first ultrasonic image when the user views the first ultrasonic image, it is possible to specify information (for example, the examination site) related to the first ultrasonic image by the processor detecting the annotation. Therefore, the ultrasonic diagnostic device can set conditions suitable for acquisition of the second ultrasonic image on the basis of information relating to the first ultrasonic image, thereby reducing the work load on the user.

An embodiment for carrying out the disclosure will be described below, but the present disclosure is not limited to the following embodiment.

### First Embodiment

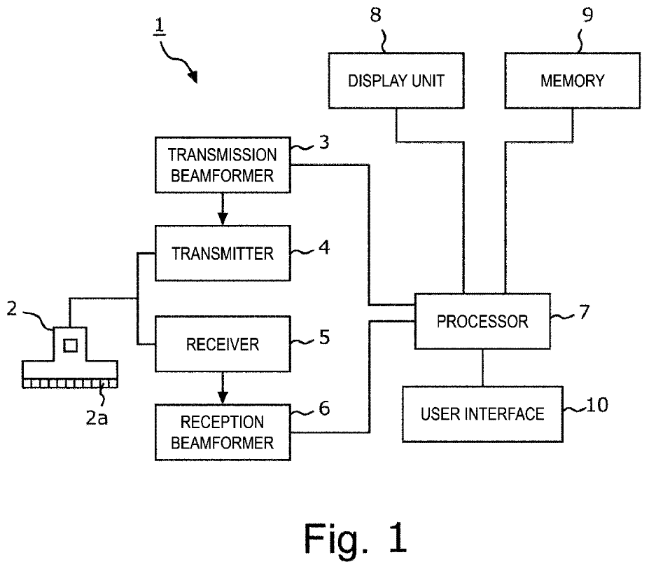

FIG. 1 is a block diagram of the ultrasonic diagnostic device 1.

The ultrasonic diagnostic device 1 has an ultrasonic probe 2, a transmission beamformer 3, a transmitter 4, a receiver 5, a reception beamformer 6, a processor 7, a display unit 8, a memory 9, and a user interface 10. The ultrasonic diagnostic device 1 is one example of the ultrasonic image display system of the present disclosure.

The ultrasonic probe 2 has a plurality of vibrating elements 2a arranged in an array. The transmission beamformer 3 and the transmitter 4 drive the plurality of vibrating elements 2a, which are arrayed within the ultrasonic probe 2, and ultrasonic waves are transmitted from the vibrating elements 2a. The ultrasonic waves transmitted from the vibrating element 2a are reflected inside the subject, and a reflection echo is received by the vibrating element 2a. The vibrating elements 2a convert the received echo to an electrical signal and output this electrical signal as an echo signal to the receiver 5. The receiver 5 executes a prescribed process on the echo signal and outputs the echo signal to the reception beamformer 6. The reception beamformer 6 executes reception beamforming on the signal received through the receiver 5 and outputs echo data.

The reception beamformer 6 may be a hardware beamformer or a software beamformer. If the reception beamformer 6 is a software beamformer, the reception beamformer 6 may include one or more processors, including one or more of: i) a graphics processing unit (GPU); ii) a microprocessor; iii) a central processing unit (CPU); iv) a digital signal processor (DSP); or v) another type of processor capable of executing logical operations. A processor configuring the reception beamformer 6 may be configured by a processor different from the processor 7 or may be configured by the processor 7.

The ultrasonic probe 2 may include an electrical circuit for performing all or a portion of transmission beamforming and/or reception beamforming. For example, all or a portion of the transmission beamformer 3, the transmitter 4, the receiver 5, and the reception beamformer 6 may be provided in the ultrasonic probe 2.

The processor 7 controls the transmission beamformer 3, the transmitter 4, the receiver 5, and the reception beamformer 6. Furthermore, the processor 7 is in electronic communication with the ultrasonic probe 2. The processor 7 controls which of the vibrating elements 2a is active and the shape of ultrasonic beams transmitted from the ultrasonic probe 2. The processor 7 is in electronic communication with the display unit 8. The processor 7 can process echo data to generate an ultrasonic image. The term “electronic communication” may be defined to include both wired and wireless communications. The processor 7 may include a central processing unit (CPU) according to one embodiment. According to another embodiment, the processor 7 may include one or more processor, another electronic constituent element that may perform a processing function such as a digital signal processor, a field programmable gate array (FPGA), a graphics processing unit (GPU), another type of processor, and the like. According to another embodiment, the processor 7 may include a plurality of electronic constituent elements capable of executing a processing function. For example, the processor 7 may include two or more electronic constituent elements selected from a list of electronic constituent elements including a central processing unit, a digital signal processor, a field programmable gate array, and a graphics processing unit.

The processor 7 may also include a complex demodulator (not illustrated in the drawings) that demodulates RF data. In another embodiment, demodulation may be executed in an earlier step in the processing chain.

Moreover, the processor 7 may generate various ultrasonic images (for example, a B-mode image, color Doppler image, M-mode image, color M-mode image, spectral Doppler image, elastography image, TVI image, strain image, and strain rate image) based on data obtained by processing via the reception beamformer 6. In addition, one or a plurality of modules can generate these ultrasonic images.

An image beam and/or an image frame may be saved and timing information may be recorded indicating when the data is retrieved to the memory. The module may include, for example, a scan conversion module that performs a scan conversion operation to convert an image frame from a coordinate beam space to display space coordinates. A video processor module may also be provided for reading an image frame from the memory while a procedure is being implemented on the subject and displaying the image frame in real-time. The video processor module may save the image frame in an image memory, and the ultrasonic images may be read from the image memory and displayed on the display unit 8.

In the present Specification, the term “image” can broadly indicate both a visual image and data representing a visual image. Furthermore, the term “data” can include raw data, which is ultrasonic data before a scan conversion operation, and image data, which is data after the scan conversion operation.

Note that the processing tasks described above handled by the processor 7 may be executed by a plurality of processors.

Furthermore, when the reception beamformer 6 is a software beamformer, a process executed by the beamformer may be executed by a single processor or may be executed by the plurality of processors.

Examples of the display unit 8 include a LED (Light Emitting Diode) display, an LCD (Liquid Crystal Display), and an organic EL (Electro-Luminescence) display. The display unit 8 displays an ultrasonic image.

The memory 9 is any known data storage medium. In one example, the ultrasonic image display system includes a non-transitory storage medium and a transitory storage medium. In addition, the ultrasonic image display system may also include a plurality of memories. The non-transitory storage medium is, for example, a non-volatile storage medium such as a Hard Disk Drive (HDD) drive, a Read Only Memory (ROM), etc. The non-transitory storage medium may include a portable storage medium such as a CD (Compact Disk) or a DVD (Digital Versatile Disk). A program executed by the processor 7 is stored in the non-transitory storage medium. The transitory storage medium is a volatile storage medium such as a Random Access Memory (RAM).

The memory 9 stores one or more commands that can be executed by the processor 7. One or more commands cause the processor 7 to execute various types of operations.

Note that the processor 7 may also be configured so as to be able to connect to an external storing device 15 by a wired connection or a wireless connection. In this case, the command(s) causing execution by the processor 7 can be distributed to both the memory 9 and the external storing device 15 for storage.

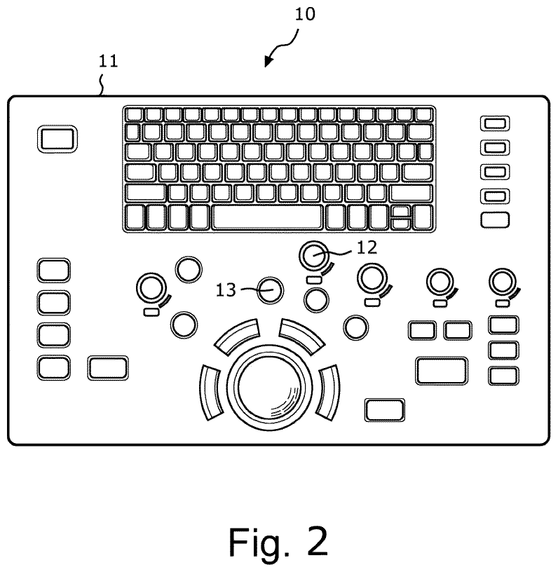

The user interface 10 can receive input from a user. For example, the user interface 10 receives instructions or information input by the user. FIG. 2 is a diagram schematically illustrating one example of the user interface 10. The user interface 10 includes an operation panel 11. The operation panel 11 includes a keyboard, hard keys, a trackball, a rotary control, soft keys, and the like. The operation panel 11 includes, for example, a CF button 12 for setting the ultrasonic diagnostic device to a color Doppler mode, and a measurement button 13 for setting the ultrasonic diagnostic device to a measurement mode. The user interface 10 may include a touch screen that displays a soft key or the like.

The ultrasonic diagnostic device 1 is configured as described above.

When an ultrasonic examination is performed using the ultrasonic diagnostic device 1, after an ultrasonic image is generated by scanning an examination site of a subject, another type of ultrasonic image may be generated by scanning the same examination site again. For example, an examination site of a subject may be scanned to generate a B-mode image, and then the same examination site may be scanned again to generate a color Doppler image. In this case, the user may set a condition for generating the color Doppler image (for example, a value of a parameter for adjusting the image quality of the color Doppler image) with reference to an organ or the like depicted in the B-mode image.

However, in this case, it is necessary for the user to perform an operation such as checking the site depicted in the B-mode image acquired in advance. The user also needs to manually input the conditions for generating the color Doppler image. Therefore, when a condition for generating a color Doppler image is set, there is a problem in that work burden on the user increases.

Therefore, the inventor of the present application has made intensive studies and devised a method capable of handling the above problems. The present method will be described below in detail.

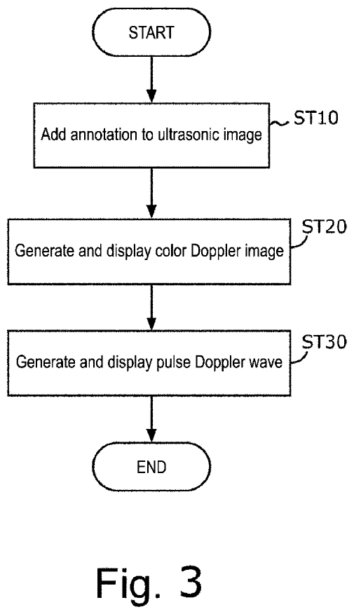

FIG. 3 is a diagram illustrating the flow in the first embodiment.

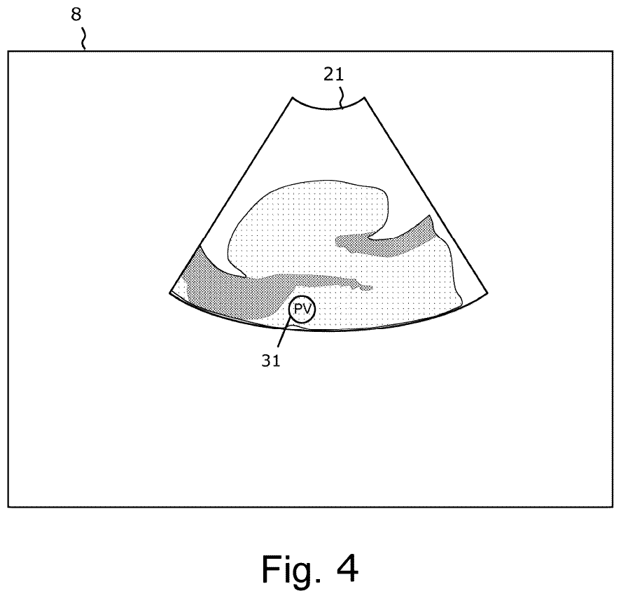

In step ST10, the user scans the examination site of the subject to acquire an ultrasonic image (for example, a B-mode image) of the examination site, and records the acquired ultrasonic image. The ultrasonic image to be recorded may be a still image or a moving image obtained by continuously capturing images during a certain period of time. Then, the user adds an annotation to the acquired ultrasonic image. FIG. 4 is an explanatory diagram of the annotation 31 added to the ultrasonic image 21. Here, an example in which the user adds “PV” as the annotation 31 to the ultrasonic image 21 is shown. “PV” stands for portal vein (portal vein). The user saves the annotation 31 (PV). After the PV is calculated, the flow proceeds to step ST20.

In step ST20, the user performs an operation of acquiring a color Doppler image of the same examination site as the ultrasonic image 21 acquired in step ST10. Step ST20 will be described below in detail.

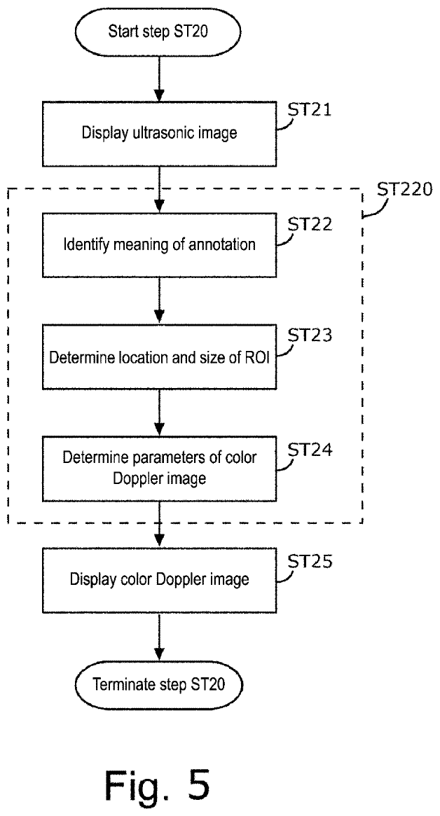

FIG. 5 is a diagram illustrating one example of the flow of step ST20.

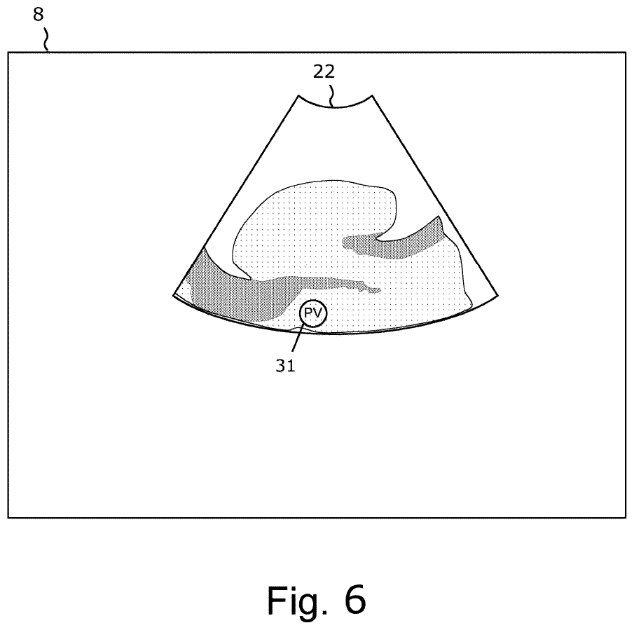

In step ST21, the user operates a probe to scan the same examination site as the examination site scanned in step ST10 again and obtains an ultrasonic image 22 different from the ultrasonic image 21 obtained in step ST10. FIG. 6 is a schematic diagram of an ultrasonic image 22 displayed on the display unit 8 by scanning in step ST21. A real-time ultrasonic image 22 (live image) or a still image may be displayed on the display unit 8. When displaying the ultrasonic image 22, the processor can also display the annotation PV stored in step ST10. Therefore, the user can confirm that the portal vein is displayed in the ultrasonic image 22 by visually recognizing the annotation PV displayed on the display unit 8. After the ultrasonic image 22 is displayed, the user presses the CF button 12 (see FIG. 2) of the user interface 10 to set the ultrasonic diagnostic device 1 to Doppler mode. When Doppler mode is set, the process proceeds to step ST220.

In step ST220, the processor controls the operation of the ultrasonic diagnostic device 1 so that a color Doppler image is generated. Specifically, the processor controls the operation of the ultrasonic diagnostic device 1 so that the position and the size of the region of interest are set and the values of the parameters of the color Doppler image are set. A specific process of step ST220 for generating the region of interest and the color Doppler image will be described below. Note that step ST220 includes steps ST22 to ST24, and thus each step will be described in order.

In step ST22, the processor identifies the meaning of the annotation PV.

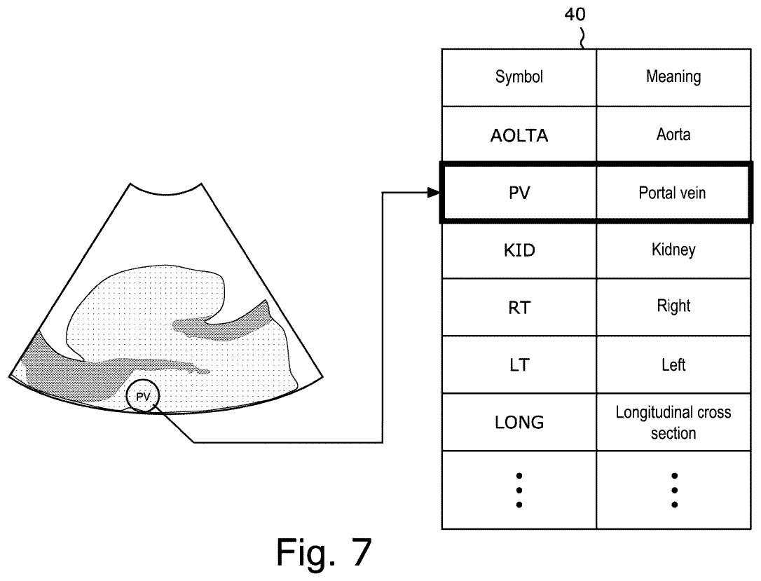

FIG. 7 is an explanatory diagram of a method for identifying the meaning of an annotation.

The processor refers to the annotation list 40 stored in the storage device and specifies the meaning of the annotation PV.

The annotation list 40 includes a “symbol” column and a “meaning” column. Characters and marks that the user is permitted to use as annotations are listed in the “symbol” column. In FIG. 7, text such as “AORTA,” “PV,” “KID,” “RT,” “LT,” and “LONG” are shown as examples of symbols permitted to be used as annotations. In the column of “meaning,” the meaning represented by the symbol is shown. For example, “AORTA” represents an artery, “PV” represents a portal vein, “KID” represents a kidney, “RT” represents right, “LT” represents left, and “LONG” represents a longitudinal cross section.

The processor specifies to which symbol among the symbols shown in the annotation list 40 the annotation added to the ultrasonic image corresponds. Here, since the annotation is “PV,” the processor specifies “PV” from the annotation list 40. Then, the processor recognizes the meaning associated with “PV.” Since “PV” is the portal vein, the processor determines that the annotation PV represents the portal vein. After identifying the meaning of the annotation, the process proceeds to step ST23.

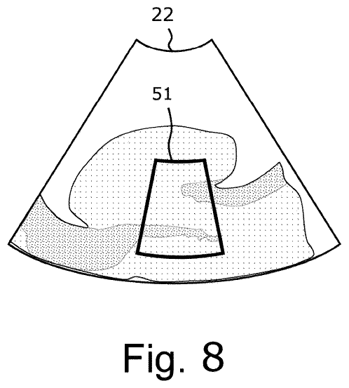

FIG. 8 is an explanatory diagram of step ST23.

In step ST23, the processor determines the position and size of the region of interest 51 with respect to the ultrasonic image 22 based on the meaning (portal vein) represented by the annotation PV specified in step ST22. Specifically, the position and the size of the region of interest 51 are determined as follows.

The annotation PV represents the portal vein. In the ultrasonic examination, the portal vein is often displayed below the central portion of the ultrasonic image 22. Therefore, in the present embodiment, when the annotation represents the portal vein, the processor determines the position and size of the region of interest 51 so that the region of interest 51 surrounds a region below the central portion of the ultrasonic image 22 as shown in FIG. 8. After the region of interest 51 and the size are set, the flow proceeds to step ST24.

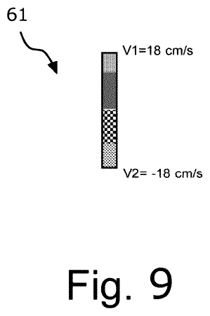

FIG. 9 is an explanatory diagram of step ST24.

The processor sets a value of a parameter for adjusting image quality of the color Doppler image. Here, as a parameter for adjusting the image quality of the color Doppler image, a flow velocity range of a color map 61 representing the flow velocity is considered.

The color map 61 represents blood flow approaching a probe in a first color and blood flow moving away from the probe in a second color. The first color is, for example, red, and the second color is, for example, blue. The difference in blood flow velocity is represented by a change in hue or a change in brightness.

The color map 61 has two flow velocity parameters V1 and V2 representing a flow velocity range. The flow velocity parameter V1 represents the maximum value of the blood flow flowing toward the displayed vessel, and the flow velocity parameter V2 represents the maximum value of the blood flow flowing in the direction away from the displayed vessel. Note that the flow velocity parameter V1 is represented by a positive value, and the flow velocity parameter V2 is represented by a negative value. The maximum value of the flow velocity is often determined by the type of organ. For example, in the portal vein, V1=18 cm/s and V2=−18 cm/s are set, in the abdominal aorta, V1=35 cm/s and V2=−35 cm/s are set, and in the kidneys, V1=12 cm/s and V2=−12 cm/s are set. Therefore, in the present embodiment, the processor sets the value of the flow velocity parameter V1 and the value of the flow velocity parameter V2 based on the input annotation. Here, since the annotation represents the portal vein, the processor sets the flow velocity parameters V1 and V2 to values suitable for the portal vein, that is, V1=18 cm/s and V2=18 cm/s. After the value of the flow velocity parameter is set, the process proceeds to step ST25.

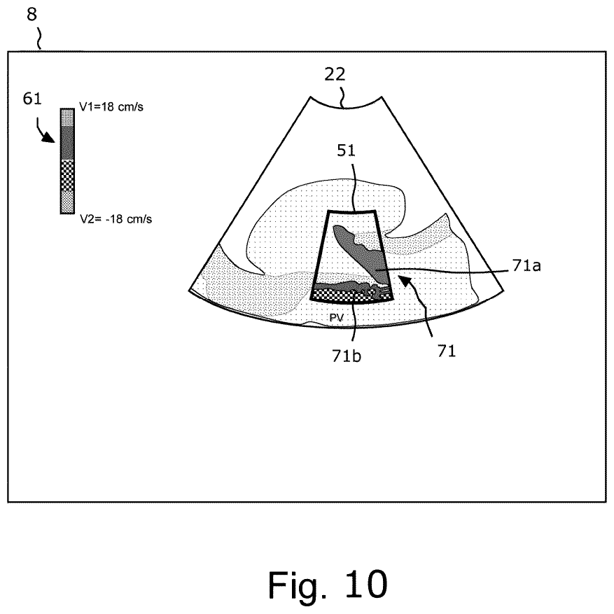

In step ST25, the processor creates a color Doppler image representing the difference in the flow velocity of the blood flow in the region of interest 51 based on the set value of the flow velocity parameter, and displays the color Doppler image. FIG. 10 schematically illustrates a color Doppler image 71 displayed on the display unit 8.

In FIG. 10, the color Doppler image 71 is displayed in the region of interest 51 set in the ultrasonic image 22 (B-mode image). A color map 61 is also displayed on the display unit 8.

A region 71a filled with gray in the region of interest 51 represents a blood flow approaching the probe, and a region 71b indicated by a black and white dot pattern represents a blood flow moving away from the probe. The user can visually recognize the direction and the flow velocity of the blood flow in the region of interest 51 by confirming which color of the color map 61 the color in the region of interest 51 displayed on the display unit 8 corresponds to. Note that, although a still image is displayed in FIG. 10, a moving image may be displayed. When the moving image is displayed, the user can visually recognize the state in which the flow velocity pattern displayed in the region 71a and the region 71b changes with time. Furthermore, the user can also manually adjust the position and size of the region of interest 51 as necessary. Therefore, even when the region of interest 51 deviates from the portal vein or the size of the region of interest 51 is too small (or too large), the user can correct the region of interest 51 to the position and/or size desired by the user. When the color Doppler image 71 is displayed, step ST20 ends, and the process proceeds to step ST30 (see FIG. 3).

In step ST30, a pulsed Doppler (PW) wave is displayed.

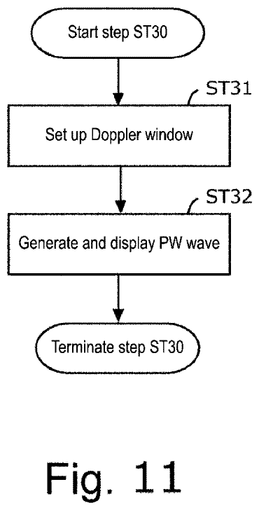

FIG. 11 is a diagram illustrating one example of the flow of step ST30.

Note that step ST30 includes steps ST31 to ST32, and thus each step will be described.

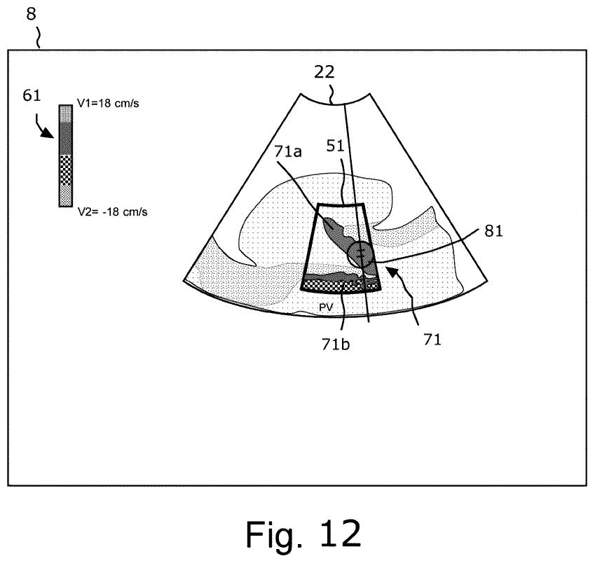

In step ST31, the user sets the Doppler window at a position where the user wants to observe the flow velocity. FIG. 12 illustrates one example of the Doppler window 81. FIG. 12 illustrates an example in which the Doppler window 81 is set in the portal vein.

Once the Doppler window 81 has been set, the process proceeds to step ST32.

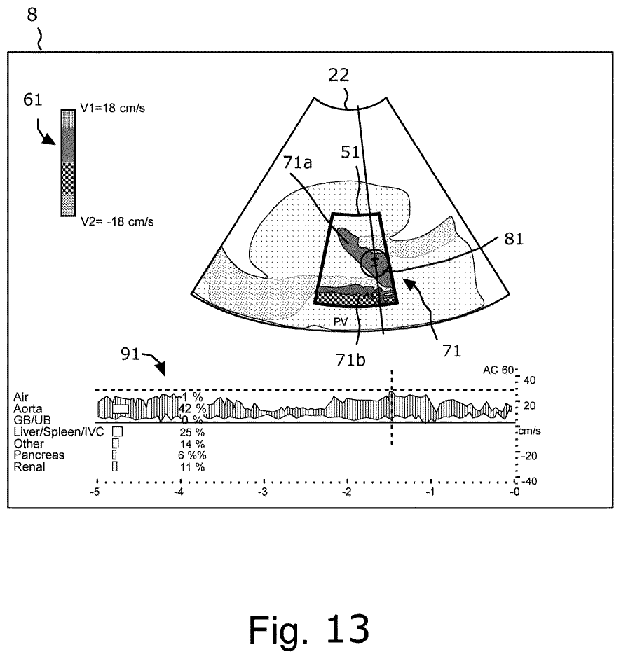

In step ST32, the processor creates a pulse Doppler (PW) waveform at the position where the Doppler window 81 is set, and displays the PW wave on the display unit 8 (see FIG. 13).

FIG. 13 is an explanatory diagram of step ST32.

When creating the PW wave 91, the processor sets the range of the PW wave 91 based on the information of the annotation. Here, since the annotation represents the portal vein, the processor sets the range of the PW wave 91 to a value suitable for the portal vein. Therefore, since a range suitable for the portal vein can be automatically set with respect to the amplitude of the PW wave 91, the folding phenomenon of the PW wave 91 can be avoided.

In the first embodiment, when creating the color Doppler image 71, the processor uses the annotation list 40 to identify that the annotation PV represents the portal vein. Therefore, the processor can set the region of interest 51 at a position substantially corresponding to the portal vein, and can reduce the burden on the user in setting the region of interest 51. For example, when the position and the size of the region of interest 51 automatically set by the processor match the position and the size of the region of interest 51 desired by the user, the user is released from the setting operation of the region of interest 51. Further, even if the position and size of the region of interest 51 are different from the position and size of the region of interest 51 desired by the user, the region of interest 51 is set at a position substantially corresponding to the portal vein by the automatic setting of the region of interest 51 by the processor. Therefore, the user can set the region of interest 51 as desired by the user only by finely adjusting the position and/or the size of the region of interest 51, so that the burden imposed on the setting operation of the region of interest 51 by the user is reduced.

In addition, as described above, since the processor specifies that the annotation PV represents the portal vein, it is possible to set the flow velocity parameters V1 and V2 to values suitable for the portal vein. Therefore, since it is not necessary for the user to manually set the flow velocity range, it is possible to reduce the work burden on the user.

Furthermore, when the PW wave 91 of the portal vein is generated, the range of the PW wave 91 is set based on the information (portal vein) represented by the annotation PV. Therefore, since a range suitable for the portal vein can be automatically set with respect to the amplitude of the PW wave 91, the PW wave 91 in which the folding phenomenon is avoided can be displayed.

### Second Embodiment

In the second embodiment, an example in which a color Doppler image is generated by combining information represented by an annotation and an image recognition algorithm will be described.

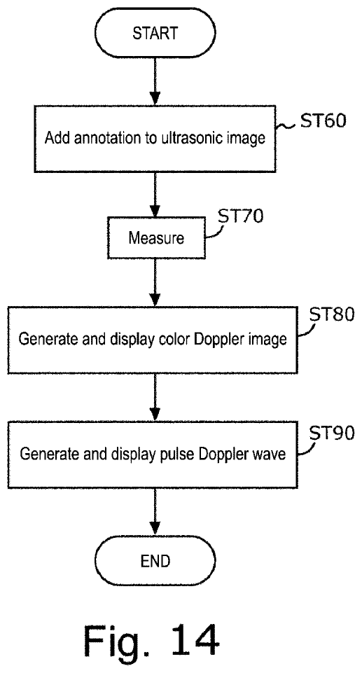

FIG. 14 is a diagram illustrating the flow in the second embodiment.

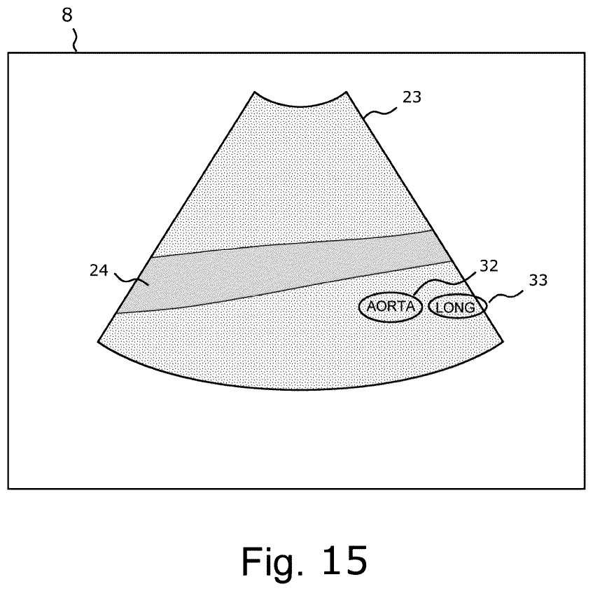

In step ST60, the user scans the examination site of the subject to acquire an ultrasonic image of the examination site, and records the acquired ultrasonic image. The ultrasonic image to be recorded may be a still image or a moving image obtained by continuously capturing images during a certain period of time. FIG. 15 is a diagram schematically illustrating an ultrasonic image 23 acquired by the scan. Here, the aorta 24 is depicted in the ultrasonic image 23. After acquiring the ultrasonic image 23, the user adds an annotation to the ultrasonic image 23. FIG. 15 illustrates an example in which two annotations 32 and 33 are added to the ultrasonic image 23. The annotation 32 is “AORTA” and the annotation 33 is “LONG.” “AORTA” represents the aorta and “LONG” represents the longitudinal section. After adding the annotations 32 and 33, the flow proceeds to step ST70.

Moreover, in step ST70, the processor measures the diameter of the aorta.

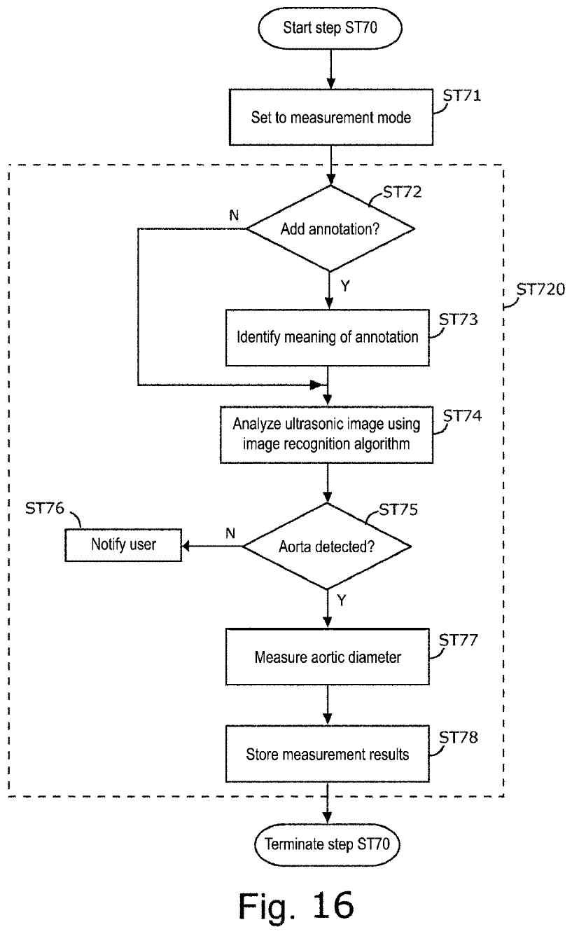

FIG. 16 is a diagram illustrating one example of the flow of step ST70.

In step ST71, the user presses the measurement button 13 (see FIG. 2) of the user interface 10 to set the ultrasonic diagnostic device 1 to the measurement mode. When the ultrasonic diagnostic device 1 is set to the measurement mode, the process proceeds to step ST720.

In step ST720, the processor controls the operation of the ultrasonic diagnostic device 1 so that detection of a measurement site from the ultrasonic image 23, measurement of the detected measurement region, and storage of the measurement result are executed. Step

ST720 will be described below in detail Note that step ST720 includes steps ST72 to ST77, and thus each step will be described.

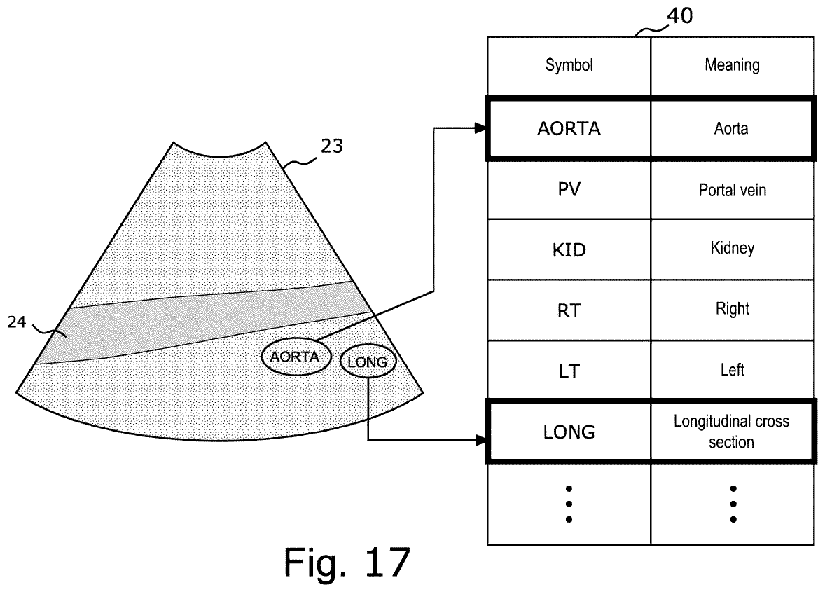

In step ST72, the processor determines whether or not an annotation is added to the ultrasonic image 23. When an annotation is added to the ultrasonic image 23, the process proceeds to step ST73. On the other hand, when the annotation is not added to the ultrasonic image 23, the process proceeds to step ST74. In the present embodiment, since the annotation is added in step ST60 (see FIG. 14), the process proceeds to step ST73. FIG. 17 is an explanatory diagram of step ST73.

The processor reads the annotation added to the ultrasonic image 23 in step ST60. Then, the processor refers to the annotation list 40 stored in the memory to specify the meaning of the annotation. In this case, the annotations added to the ultrasonic image 23 are “AORTA” and “LONG.” First, the processor specifies “AORTA” from the annotation list 40, and recognizes that the meaning associated with “AORTA” is the aorta. Further, the processor specifies “LONG” from the annotation list 40 and recognizes that the meaning associated with “LONG” is “longitudinal cross section.” Therefore, the processor determines that the annotation input by the user represents “aorta” and “longitudinal cross section.” After identifying the meanings of the annotations, the process proceeds to step ST74.

In step ST74, the processor analyzes the ultrasonic image 23 using an image recognition algorithm. The image recognition algorithm may be an algorithm using an Al technique (a deep learning model or the like) or an algorithm using a technique (for example, a segmentation method) different from the Al technique. After analyzing the ultrasonic image 23, the process proceeds to step ST75.

In step ST75, the processor determines whether the measurement site represented by the annotation is detected in the ultrasonic image 23 by analysis of the ultrasonic image 23. Here, it is determined whether an aorta 24 is detected as the measurement site. If the aorta is detected, the process proceeds to step ST77.

On the other hand, if the aorta is not detected or if a site other than the aorta is detected, it is conceivable that there is an error in the content of the annotation. Therefore, if the aorta is not detected or if a site other than the aorta is detected, the process proceeds to step ST76, and the user is notified that there is a possibility that the annotation is incorrect. Upon receiving this notification, the user can check the annotation and, as necessary, scan the examination site of the subject again.

Here, the aorta 24 is depicted in the ultrasonic image 23, and thus the processor detects the aorta. Therefore, the process proceeds to step ST77.

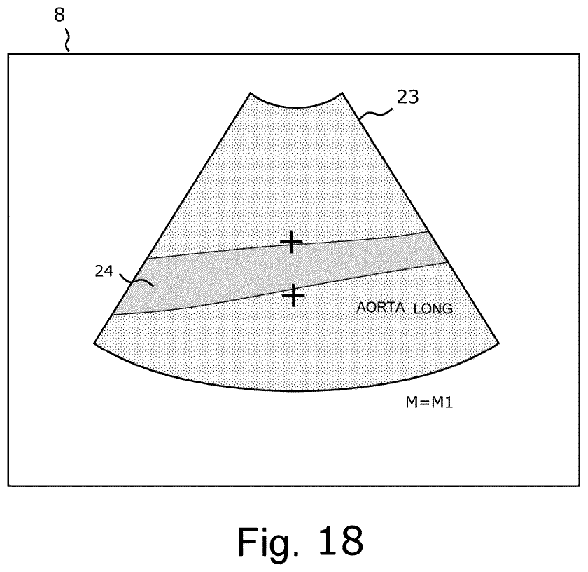

Moreover, in step ST77, the processor measures the diameter of the aorta. FIG. 18 is a diagram illustrating a position where the diameter of the aorta is measured. The processor has detected the aorta 24 from the ultrasonic image 23, and thus position information of where the aorta is present relative to the ultrasonic image 23 is known. Therefore, the processor can set any position in the longitudinal direction of the aorta 24 as a measurement position and measure the diameter of the aorta. FIG. 18 illustrates a state in which the diameter of the aorta is measured, having a central portion in the longitudinal direction of the aorta 24 as the measurement position. When the diameter of the aorta is measured, a measurement value M (=M1) is displayed on the display unit 8. Once the measurement value is measured, the process proceeds to step ST78.

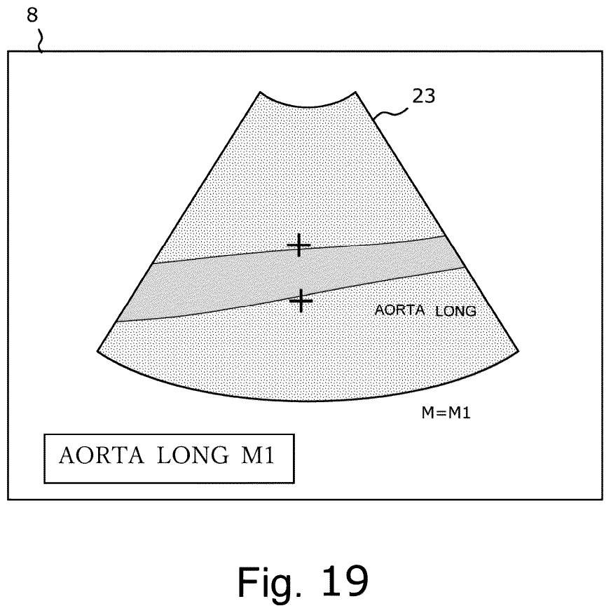

In step ST78, the processor stores the measurement result based on the annotation. FIG. 19 is a schematic diagram of one example of measurement results. The measurement result includes three items “measurement site,” “measurement cross-section,” and “measurement value.” In FIG. 19, the measurement site is “AORTA,” the measurement cross-section is “LONG,” and the measurement value is “M1.” The annotations are “AORTA” and “LONG,” and thus the processor can recognize that the measurement site is “AORTA” (that is, aorta) and the measurement cross-section is “LONG” (that is, longitudinal cross-section). The processor also measures the diameter of the aorta (M=M1) at step ST77. Accordingly, the processor can create a measurement result including the measurement site “AORTA,” the measurement cross-section “LONG,” and the measurement value “M1.” Once the measurement result is created, the processor stores the measurement result in the storage device and ends the measurement step of step ST70.

Note that, in the second embodiment, it is determined in step ST72 that an annotation has been added. However, there are cases where no annotation is added. In this case, in step ST72, the processor determines that an annotation has not been added, and thus skips step ST73 from step ST72 and proceeds to step ST74. Therefore, the processor cannot use the information of the annotation but can analyze the ultrasonic image 23 using the image recognition algorithm, and thus the processor can perform the measurement of the aorta based on the analysis result of the ultrasonic image 23 and store the measurement result.

Once the measurement complete, the process proceeds to step ST80 (see FIG. 14).

In step ST80, the user performs an operation of acquiring a color Doppler image 71 of the same examination site as the examination site scanned in step ST60. Step ST80 will be described below in detail.

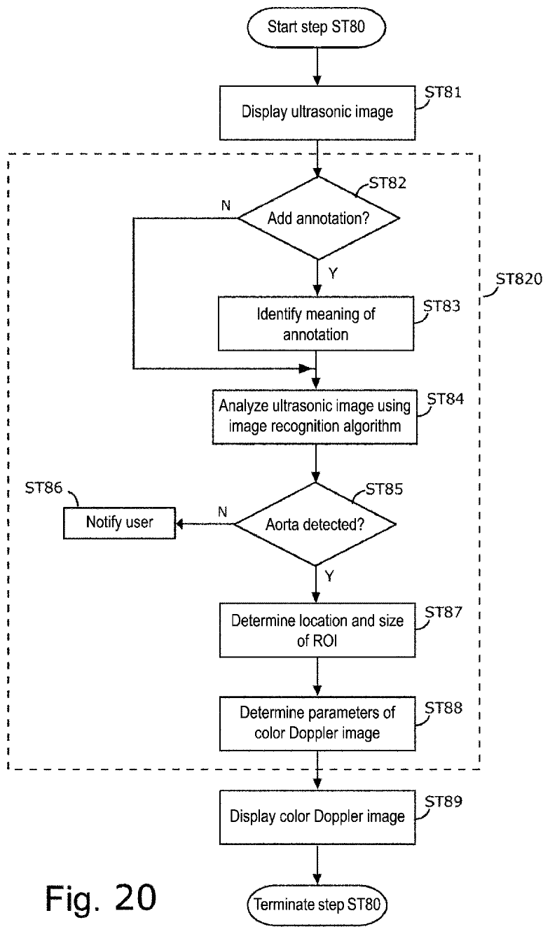

FIG. 20 is a diagram illustrating one example of the flow of step ST80.

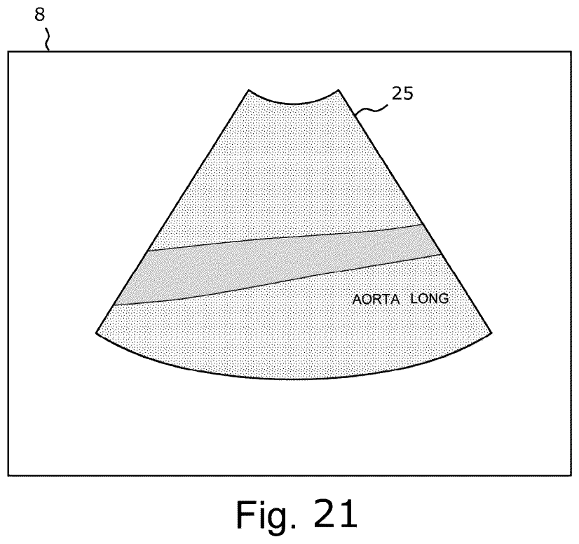

In step ST81, the user operates the probe to scan the same examination site as the examination site scanned in step ST60 again, and obtains an ultrasonic image 25 different from the ultrasonic image 23 obtained in step ST60. FIG. 21 is a schematic diagram of an ultrasonic image 25 displayed on the display unit 8 by scanning in step ST81. A real-time ultrasonic image 25 (live image) or a still image may be displayed on the display unit 8. Furthermore, when displaying the ultrasonic image 25, the processor can also display the annotations “AORTA” and “LONG” stored in step ST60. Therefore, the user can confirm that the aorta is displayed in the ultrasonic image 25 by visually recognizing the annotations “AORTA” and “LONG” displayed on the display unit 8. After the ultrasonic image 25 is displayed, the user presses a CF button 12 (see FIG. 2) of a user interface 10 to set the ultrasonic diagnostic device 1 to a Doppler mode. When the Doppler mode is set, the process proceeds to step ST820.

In step ST820, the processor generates a color Doppler image by controlling the operation of the ultrasonic diagnostic device 1 so that image quality suitable for the color Doppler image is realized. Specifically, the processor sets a position and a size of the region of interest, and further sets a value of a parameter of the color Doppler image. A specific process of step ST820 for generating the region of interest and the color Doppler image will be described below. Note that step ST820 includes steps ST82 to ST86, and thus each step will be described in order.

In step ST82, the processor determines whether an annotation has been added to the ultrasonic image 23 (see FIG. 15) acquired in step ST60. Here, the annotations “AORTA” and “LONG” are added to the ultrasonic image 23, and thus the process proceeds to step ST83.

In step ST83, the processor identifies the meaning of the annotations “AORTA” and “LONG.” Note that the method of identifying the meaning of the annotation is the same as that in step ST73, and thus description thereof will be omitted. The processor determines that the annotation represents “aorta” and “longitudinal cross-section.” After identifying the meanings of the annotations, the process proceeds to step ST84.

In step ST84, the processor analyzes the ultrasonic image 25 using an image recognition algorithm. After analyzing the ultrasonic image 25, the process proceeds to step ST85.

In step ST85, the processor determines whether the aorta is detected from the ultrasonic image 25 by analysis of the ultrasonic image 25. If the aorta is detected, the process proceeds to step ST87.

On the other hand, if the aorta is not detected, it is considered that there is an error in the content of the annotation. Therefore, if the aorta is not detected, the process proceeds to step ST86 to notify the user that there is a possibility that the annotation is incorrect. Upon receiving this notification, the user can check the annotation and, as necessary, scan the examination site of the subject again.

Here, the aorta is depicted in the ultrasonic image 25, and thus the processor detects the aorta. Therefore, the process proceeds to step ST87.

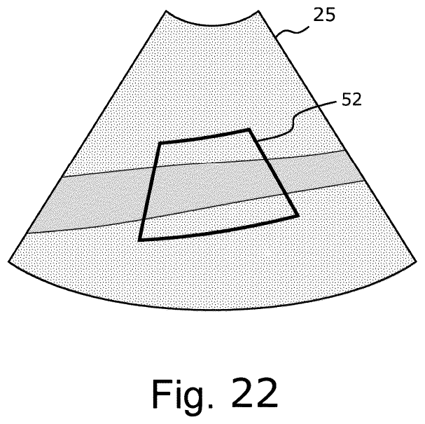

FIG. 22 is an explanatory diagram of step ST87.

The processor analyzes the ultrasonic image 25 using an image recognition algorithm to detect the aorta in the ultrasonic image 25. Therefore, the processor can recognize the position information of where the aorta is present relative to the ultrasonic image 25, and thus a region of interest 52 can be set at any position of the aorta. Here, the processor determines the position and size of the region of interest 52 such that the region of interest 52 surrounds the central portion of the aorta as illustrated in FIG. 22. In addition, the processor recognizes the position information of where the aorta is present relative to the ultrasonic image 25, and thus it is also possible to set an incline of the region of interest 52 so as to be along the running direction of the aorta. Once the region of interest 52 is set, the process proceeds to step ST88.

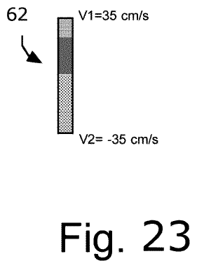

FIG. 23 is an explanatory diagram of step ST88.

The processor sets a value of a parameter for adjusting image quality of the color Doppler image. Here, as a parameter for adjusting the image quality of the color Doppler image, a flow velocity range of a color map 62 representing the flow velocity is considered.

As in the first embodiment, the processor sets a value of a first flow velocity parameter V1 and a value of a second flow velocity parameter V2 based on the annotation. Here, the annotation represents the aorta, the processor sets V1 and V2 to values suitable for the aorta, that is, V1=35 cm/s and V2=−35 cm/s. After the value of the flow velocity parameter is set, the process proceeds to step ST89.

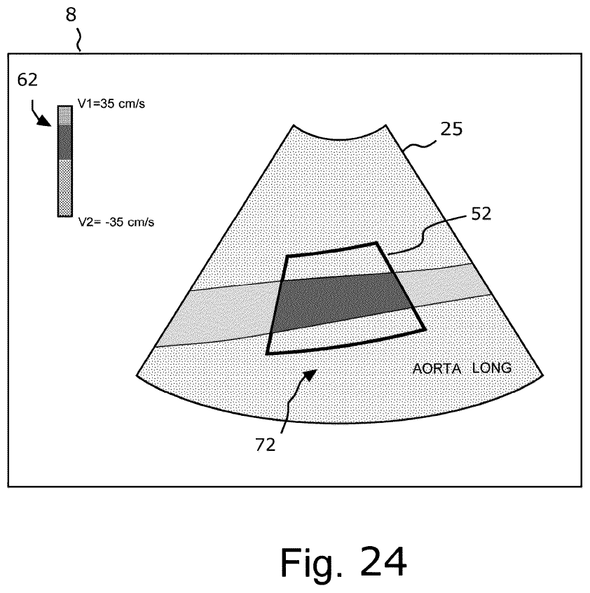

In step ST89, the processor creates a color Doppler image representing the difference in the flow velocity of the blood flow in the region of interest 52 based on the set value of the flow velocity parameter, and displays the color Doppler image. FIG. 24 schematically illustrates a color Doppler image 72 displayed on the display unit 8. Once the color Doppler image 72 is displayed, step ST80 ends.

Note that, in the second embodiment, it is determined in step ST82 that an annotation has been added. However, there are cases where no annotation is added. In this case, in step ST82, the processor determines that an annotation has not been added, and thus skips step ST83 from step ST82, and proceeds to step ST84. Therefore, the processor cannot use the information of the annotation but can analyze the ultrasonic image 25 using the image recognition algorithm, and thus the processor can detect the aorta based on the analysis result of the ultrasonic image 25 and can execute the processing of steps ST87 and ST88.

Once the color Doppler image 72 is displayed, the process proceeds to step ST90 (see FIG. 14).

In step ST90, a pulse Doppler (PW) wave is displayed.

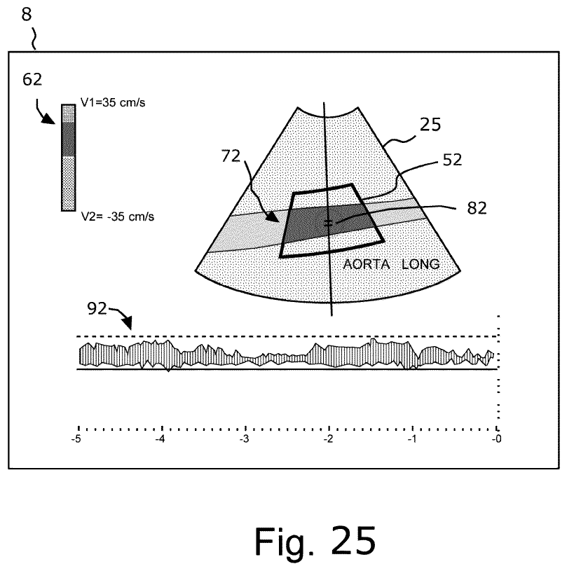

FIG. 25 is an explanatory diagram of step ST90.

In step ST90, the processor positions a Doppler window 82. The processor recognizes the position information of the aorta, and thus the Doppler window 82 can be set at any position of the aorta. For example, the processor may set the Doppler window 82 in the central portion of the aorta. Then, the processor creates a pulse Doppler (PW) waveform at the position where the Doppler window 82 is set, and displays a PW wave 92 on the display unit 8.

When creating the PW wave 92, the processor sets a range of the PW wave 92 based on the information of the annotation. Here, the annotation represents the aorta, and thus the processor sets the range of PW waves 92 to a value suitable for the aorta. Therefore, a range suitable for the aorta can be automatically set for the amplitude of the PW wave 92, and thus a folding phenomenon of the PW wave 92 can be avoided.

Note that, when necessary, the user can finely adjust the position of the Doppler window 82 automatically set by the processor. For example, when the user moves the Doppler window 82 to another location, the processor displays the PW wave 92 corresponding to the other location. Therefore, the user can display the PW wave 92 at a desired position in the aorta.

As described above, in the second embodiment, when the diameter of the aorta is measured in step ST70, the processor stores the measurement result based on the annotation. The measurement result includes three items “measurement site,” “measurement cross-section,” and “measurement value.” The processor can identify the measurement site and the measurement cross-section from the information of the annotation, and thus it is possible to prevent the user from manually inputting the data of the measurement site, the measurement cross-section, and the measurement value, thereby reducing the work load on the user.

Furthermore, in the second embodiment, when the color Doppler image 72 is generated in step ST80, the processor identifies the meanings of the annotations “AORTA” and “LONG” and performs image analysis on the ultrasonic image 25 using an image recognition algorithm. Thus, the processor can automatically detect the aorta from within the ultrasonic image 25. In addition, the processor detects the aorta from the ultrasonic image 25, and thus position information of where the aorta is present relative to the ultrasonic image 25 is known. Therefore, the processor can set the region of interest 52 at any position in the aorta. When the initial position of the region of interest 52 set by the processor coincides with the desired position considered by the user, it is not necessary for the user themselves to adjust the position of the region of interest 52, and thus the user can skip the setting operation of the region of interest 52 and start the next operation.

Note that, depending on the position at which the region of interest 52 is automatically set, the user may wish to adjust the position of the region of interest 52. In this case, the user needs to fine-tune the position of the region of interest 52. However, in the second embodiment, the region of interest 52 is automatically set by the processor so as to surround the central portion of the aorta, the user can set the region of interest 52 at a desired position only by moving the region of interest 52 from the central portion of the aorta. Therefore, the user can set the region of interest 52 at a desired position without largely changing the position of the region of interest 52, and thus it is possible to reduce the work load of the user when setting the region of interest 52.

Further, in the second embodiment, the processor identifies the meaning (aorta) of the annotation “AROTA.” Therefore, when the ultrasonic image 25 is analyzed using the image recognition algorithm, the aorta depicted in the ultrasonic image 25 is prevented from being erroneously recognized as another blood vessel. For example, the aorta is similar in morphology to the inferior vena cava (IVC), and it may be difficult to distinguish between the aorta and the IVC using image recognition algorithms alone. However, in the second embodiment, it is known from the annotation that the blood vessel on the ultrasonic image 25 is the aorta. Therefore, the processor can reliably set the flow velocity parameters V1 and V2 to values suitable for the aorta, and thus it is possible to prevent the flow velocity parameters V1 and V2 from being set to values corresponding to the inferior vena cava (IVC).

Furthermore, when creating the PW wave 92 of the aorta, the processor sets a range of the PW wave 92 based on the information of the annotation. Therefore, it is possible to avoid the range of the vertical axis of the PW wave 92 being set to a range corresponding to a blood vessel other than the aorta, and thus it is possible to avoid a decrease in the folding of the PW wave 92.

### Third Embodiment

Note that, in the second embodiment, an example in which the measurement button is provided on the operation console is described, but in the third embodiment, an example in which two measurement buttons (a measurement button for manual measurement and a measurement button for automatic measurement) are provided on an operation console will be described.

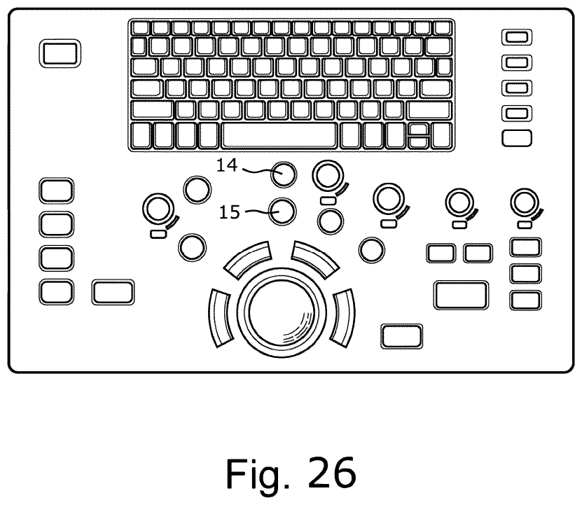

FIG. 26 is a schematic diagram of the operation console of the ultrasonic diagnostic device 1 according to the third embodiment.

The operation console is provided with a first measurement button 14 and a second measurement button 15.

The first measurement button 14 is a manual measurement button for the user to manually perform measurement. On the other hand, the second measurement button is the automatic measurement button 15, for automatically performing measurement.

A method of performing measurement using the ultrasonic diagnostic device 1 provided with the two measurement buttons will be described below. Note that, in comparison with the second embodiment, the third embodiment is different in step ST70, but other steps are the same as those in the second embodiment. Therefore, below, the main items described will be the measurement steps.

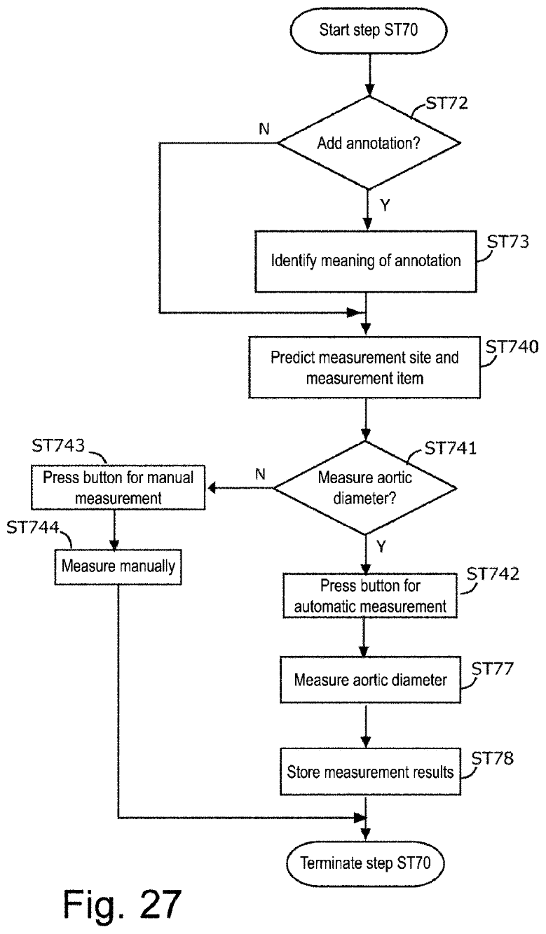

FIG. 27 is one example of a flowchart of measurement steps in the third embodiment.

Steps ST72 and ST73 are the same as in the flow of the second embodiment in FIG. 16, and thus description is omitted. The processor determines that the annotation input by the user represents “aorta” and “longitudinal cross-section.” After identifying the meanings of the annotations, the process proceeds to step ST74.

In step ST740, the processor predicts the measurement site and the measurement item based on the meaning of the annotation. In the present embodiment, the annotations are “aorta” and “longitudinal cross-section,” and thus the processor predicts the measurement site as “aorta” and predicts the measurement item as “diameter of aorta.” After predicting the measurement site and the measurement item, the process proceeds to step ST741.

In step ST741, the processor confirms with the user whether to measure the diameter of the aorta. By displaying, for example, a notification of “Measure the diameter of the aorta?” on the display unit 8, it is possible to confirm with the user. When it is determined that the diameter of the aorta is to be measured, the user proceeds to step ST742 and presses the automatic measurement button 15. When this button is pressed, the processor determines that the diameter of the aorta is automatically measured. Then, the diameter of the aorta is automatically measured in step ST77, the measurement result is automatically stored in step ST78, and the flow ends.

On the other hand, when it is determined that the user wants to measure not the diameter of the aorta but another site, the process proceeds to step ST743, and the user presses a manual measurement button 14. When this button is pressed, the processor determines that automatic measurement will not be performed. Then, the process proceeds to step ST744, where the user manually performs measurement, and the flow ends.

In the third embodiment, the user is asked whether to measure the diameter of the aorta in step ST741. Therefore, when the measurement site and the measurement item predicted by the processor match the measurement site and the measurement item intended by the user, the measurement can be automatically performed by pressing the automatic measurement button. Furthermore, when the measurement site and the measurement item predicted by the processor do not match the measurement site and the measurement item intended by the user, the measurement can be manually performed by pressing the manual measurement button. Therefore, even when the prediction of the processor is incorrect, the measurement intended by the user can be performed.

### DESCRIPTION OF CODES

1 Ultrasonic diagnostic device
2 Ultrasonic probe
2a Vibrating element
3 Transmission beamformer
4 Transmitter
5 Receiver
6 Reception beamformer
7 Processor
8 Display unit
9 Memory
10 User interface
11 Operation panel
12 CF button
13 Measurement button
21, 22, 23, 25 Ultrasonic image
24 Aorta
31, 32, 33 Annotation
40 Annotation list
51, 52 Region of interest
61, 62 Color map
71, 72 Color Doppler image
71a, 71b Region
82 Doppler window
91, 92 PW wave

What is claimed is:

1. An ultrasonic diagnostic device comprising one or more processors for controlling operation of the ultrasonic diagnostic device for acquiring a second ultrasonic image of the same examination site as a first ultrasonic image based on an annotation added to the first ultrasonic image.

2. The ultrasonic diagnostic device according to claim 1, wherein controlling the operation of the ultrasonic diagnostic device includes at least one of adjusting image quality of the second ultrasonic image and determining a position and a size of a region of interest of the second ultrasonic image.

3. The ultrasonic image according to claim 2, wherein the one or more processors execute determining whether the first ultrasonic image has had an annotation added thereto and when the first ultrasonic image has had an annotation added thereto, controlling operation of the ultrasonic diagnostic device to acquire the second ultrasonic image of the same examination site as the first ultrasonic image based on the annotation and an image recognition algorithm that analyzes the ultrasonic image.

4. The ultrasonic diagnostic device of claim 3, wherein, when the first ultrasonic image does not have an annotation added thereto, the one or more processors control operation of the ultrasonic diagnostic device to acquire the second ultrasonic image of the same examination site as the first ultrasonic image based on the image recognition algorithm.

5. The ultrasonic diagnostic device according to claim 2, wherein:
the ultrasonic diagnostic device acquires the first ultrasonic image by scanning an examination site of a subject, and then acquires another ultrasonic image different from the first ultrasonic image by scanning the same examination site as the examination site again; and
the one or more processors execute:
analyzing the other ultrasonic image using the image recognition algorithm;
determining whether the region represented by the annotation is detected from the other ultrasonic image by analyzing the other ultrasonic image;
determining a position and a size of the region of interest when the site represented by the annotation is detected;
determining a value of a parameter of the second ultrasonic image; and
generating the second ultrasonic image in the region of interest based on the value of the parameter.

6. The ultrasonic diagnostic device according to claim 5, wherein the second ultrasonic image is a color Doppler image, and the parameter is a flow velocity parameter.

7. The ultrasonic diagnostic device according to claim 5, wherein, when the image recognition algorithm detects a site other than the examination site represented by the annotation, the image recognition algorithm notifies a user that the annotation may be incorrect.

8. The ultrasonic diagnostic device of claim 3, wherein the one or more processors execute identification of a meaning of the annotation.

9. The ultrasonic diagnostic device according to claim 8, wherein the one or more processors identify the meaning of the annotation added to the first ultrasonic image using an annotation list including symbols permitted to be used as annotations and meanings represented by the symbols.

10. The ultrasonic diagnostic device of claim 3, wherein the image recognition algorithm uses a deep learning model.

11. The ultrasonic diagnostic device according to claim 1, wherein the annotation includes information representing an examination site.

12. The ultrasonic diagnostic device according to claim 2, wherein the one or more processors control operation of the ultrasonic diagnostic device such that the following are executed:
detecting a measurement site from the first ultrasonic image;
performing a measurement of the detected measurement site; and
storing measurement results.

13. The ultrasonic diagnostic device according to claim 12, wherein the measurement result includes a measurement site, a measurement cross-section, and a measurement value.

14. The ultrasonic diagnostic device according to claim 12, wherein the one or more processors execute:
determining whether an annotation is added to the first ultrasonic image; and
when an annotation is added to the first ultrasonic image, detecting a site represented by the annotation from the first ultrasonic image as a measurement site.

15. The ultrasonic diagnostic device according to claim 14, wherein the one or more processors execute:
analyzing the first ultrasonic image using an image recognition algorithm; and
determining whether the region represented by the annotation is detected from the first ultrasonic image by analysis of the first ultrasonic image.

16. The ultrasonic diagnostic device according to claim 15, wherein the one or more processors notify the user that there is a possibility that the annotation is incorrect when the region indicated by the annotation is not detected from the first ultrasonic image or when a region different from the region indicated by the annotation is detected.

17. The ultrasonic diagnostic device according to claim 12, wherein the ultrasonic diagnostic device includes a manual measurement button and an automatic measurement button, and the one or more processors execute:
predicting a measurement item based on the annotation;
confirming with a user whether to measure the measurement item; and
determining that the measurement item is to be automatically measured when the automatic measurement button is pressed, but determining that the measurement item is not to be automatically measured when the manual measurement button is pressed.

18. The ultrasonic diagnostic device according to claim 17, wherein the measurement item is a diameter of a blood vessel.

19. The ultrasonic diagnostic device according to claim 1, wherein the first ultrasonic image is a B-mode image, and the second ultrasonic image is a color Doppler image.

20. A non-transitory computer readable storage medium in which a command is stored, wherein the command, when executed by one or more processors, causes the one or more processors to execute controlling an operation of the ultrasonic diagnostic device for acquiring a second ultrasonic image of the same examination site as that of a first ultrasonic image based on an annotation added to the first ultrasonic image.
